(19) United States
(12) Patent Application Publication
(10) Pub. No.: US 2026/0316763 A1
(43) Pub. Date: Oct. 15, 2026

(54) Sensing Platform For Dynamic Soilproperties Characterization In Autonomous Vehicles Attitude Control

(71) Applicant: Board of Trustees of Michigan State University, East Lansing, MI (US)

(72) Inventors: Nizar LAJNEF, East Lansing, MI (US); Mahmood HAQ, Okemos, MI (US); Satish UDPA, Okemos, MI (US)

(21) Appl. No.: 19/467,900

(22) Filed: Feb. 3, 2026

Related U.S. Application Data
(60) Provisional application No. 63/760,211, filed on Feb. 19, 2025.

(51) Int. Cl. B60W 40/06; B60W 10/12; B60W 10/30; B60W 50/00; G01L 5/00; G01N 3/34; G01N 33/24; G01P 1/02

(52) U.S. Cl. CPC B60W 40/06; B60W 10/12; B60W 10/30; G01L 5/0004; G01N 3/34; G01N 33/246; G01P 1/023; B60W 2050/0095; B60W 2420/22; B60W 2420/905; B60W 2710/125; B60W 2710/30; G01N 2203/0005; G01N 2203/0025; G01N 2203/0037; G01N 2203/0244

(57) ABSTRACT

A rotating penetrometer for use in soil includes an actuator arm, a hollow wheel having an outer wall, a circuit board disposed within the hollow wheel, a plurality of strain sensors disposed on the circuit board generating strain signals, an accelerometer disposed on the circuit board generating an acceleration signal, a gyroscopic sensor generating an orientation signal, a load cell coupled to the actuator arm and a moisture sensor. One or two rotating penetrometer may be used to help provide soil feedback to control a vehicle.

### CROSS-REFERENCE TO RELATED APPLICATION

This application claims the benefit of U.S. Provisional Application 63/760,211, filed on Feb. 19, 2025. The entire disclosure of the above application is incorporated herein by reference.

### GOVERNMENT FUNDING

This invention was made with government support under W56HZV-19-2-0001 awarded by the U.S. Army. The government has certain rights in the invention.

### FIELD

The present disclosure relates to attitude control for a vehicle and, more specifically, to a sensing platform to control attitude.

### BACKGROUND

This section provides background information related to the present disclosure which is not necessarily prior art.

Autonomous vehicles (AVs) rely on sensors such as global navigation satellite systems (GNSS), light detection and ranging (LiDAR), and inertial measurement units (IMUs) to navigate and perceive their environment. However, little attention has been given to the interaction between vehicles and terrain, particularly on uneven surfaces where soil properties play a critical role. Variations in shear strength, bearing capacity, and soil deformation significantly affect vehicle performance, especially under dynamic conditions.

On rough terrains, road profile irregularities introduce challenges that go beyond standard suspension adjustments. To ensure reliable autonomous operation, there is a pressing need to incorporate real-time terrain data into vehicle control algorithms. By doing so, stability and maneuverability can be actively optimized in response to changing soil conditions.

### SUMMARY

This section provides a general summary of the disclosure, and is not a comprehensive disclosure of its full scope or all of its features.

The present disclosure provides a sensor platform designed to address the needs for improved an improved sensing system and control. The sensing system measures key soil parameters such as shear modulus, shear strength, and failure limits, feeding real-time data into vehicle control algorithms. These insights will allow for the back-calculation of maneuvering forces acting on the vehicle and enable advanced control strategies to dynamically adjust suspension systems for improved stability. By integrating this sensor platform into vehicle control systems, the project aims to bridge the gap between terrain interaction and vehicle performance, ensuring safe and efficient autonomous navigation on challenging terrains.

The present disclosure addresses the problem of soil-vehicle interaction with the goal of determining soil properties in real time during vehicle operation. The work emphasizes the creation of an integrated system that combines advanced sensing hardware with computational tools to enhance vehicle stability and performance on varying terrains.

In one aspect of the disclosure, a sensing apparatus for attachment to a vehicle is set forth. The apparatus is equipped with strain and force sensors to extract critical soil properties such as shear modulus, shear strength, and bearing capacity during operation. The system is intended to operate reliably under dynamic conditions and provide high-resolution, real-time data for integration into vehicle control algorithms.

In another aspect, a computationally efficient model is developed as firmware to process real-time sensor data onboard the vehicle. This model is designed to back-calculate soil properties from strain and force measurements at a rate of at least 100 computations per second. The processed data is used to support dynamic control adjustments, ensuring stability and maneuverability on challenging terrain.

In another aspect, a computational method is developed to verify experimental data collected by the sensing apparatus. This method is also used to generate synthetic data for a wide range of soil types and conditions that cannot be practically tested experimentally. By combining experimental and simulated data, a comprehensive database of soil properties is created to support model training and validation.

In another aspect, a rotating penetrometer for use in soil includes an actuator arm, a hollow wheel having an outer wall, a circuit board disposed within the hollow wheel, a plurality of strain sensors disposed on the circuit board generating strain signals, an accelerometer disposed on the circuit board generating an acceleration signal, a gyroscopic sensor generating an orientation signal, a load cell coupled to the actuator arm and a moisture sensor. One or two rotating penetrometer may be used to help provide soil feedback to control a vehicle.

In another aspect of the disclosure, a vehicle includes a first rotating penetrometer, a second rotating penetrometer, a soil property determination system coupled to the first rotating penetrometer and the second rotating potentiometer and determining a first bearing capacity and a first shear strength associated with the first rotating penetrometer, and a second bearing capacity and a second shear strength associated with the second rotating penetrometer. A vehicle system and a vehicle controller are coupled to the soil property determination system and the vehicle system. The vehicle controller receives the first bearing capacity, the first shear strength, the second bearing capacity and the second shear strength. The vehicle controller generates a control signal for controlling the vehicle system based on the first bearing capacity and the second bearing capacity of the soil.

In another aspect of the controller, a method of controlling a vehicle includes rotating a penetrometer on the soil, generating sensor signals from the penetrometer during rotating, determining a sheer strength and bearing capacity based on the sensor signals and controlling a vehicle system based on the bearing capacity and shear strength.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1A is a first example of a traditional penetrometer.

FIG. 1B is a second example of a traditional penetrometer.

FIG. 1C is a third example of a traditional penetrometer.

FIG. 2 is a plot for determining soil behavior by comparing the tip or cone resistance to friction ratio.

FIG. 3 is a diagram showing bearing capacity and sheer strength formulas in the derivation thereof.

FIG. 4 is a side view of a wheel used as a penetrometer.

FIG. 5 is a top view of the wheel of FIG. 4.

FIG. 6A is a perspective view of an example of the wheel.

FIG. 6B is a perspective view of the wheel having a hollow interior.

FIG. 7 is a block diagrammatic view of the soil property determination system.

FIG. 8A is a first example of a flowchart for controlling a vehicle according to the bearing capacity.

FIG. 8B is a flowchart for controlling a vehicle having two different soil classification systems at either side of the vehicle.

FIG. 9A is a perspective view of a vehicle having the soil determination system incorporated therein.

9B is a top view of a vehicle have two penetrometers.

FIG. 10A is a plot of strain versus time for organic soil.

FIG. 10B is a plot of strain versus time for sandy soil.

FIG. 10C is a plot of strain versus time for gravel and dirt.

FIG. 11 is a diagram of a physical model of the interaction of soil particles.

FIG. 12A is a plot of penetration resistance versus penetration depth for sandy loam soil.

FIG. 12B is a plot of penetration resistance versus penetration depth for clay and silt soil.

FIG. 13A is a diagrammatic representation of neural networks used to determine shear strength.

FIG. 13B is a diagrammatic representation of neural networks that are used to obtain bearing capacity.

Corresponding reference numerals indicate corresponding parts throughout the several views of the drawings.

### DETAILED DESCRIPTION

Example embodiments will now be described more fully with reference to the accompanying drawings.

A research approach to the challenges of soil model complexity and the need for real-time sensing and calculations using noisy data under field conditions is address in the present system. The present system improves upon standard Dynamic Cone Penetration (DCP) testing devices as described below. Known testing is done by proving into the soil while the vehicle is stationary and not suitable for a moving vehicle.

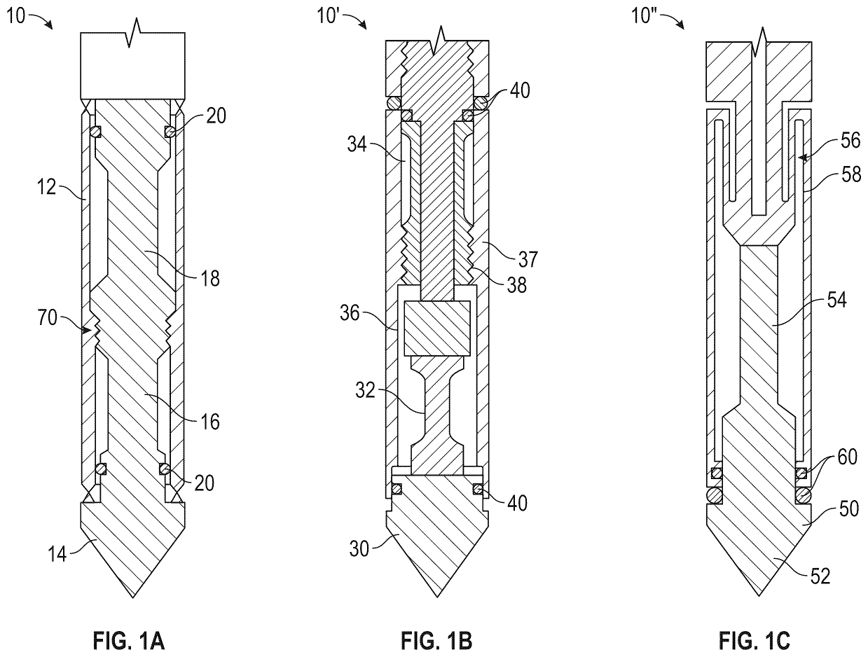

Referring now to FIGS. 1A, 1B and 1C, examples of three different cone penetrometers are illustrated. In FIG. 1A, a penetrometer 10 includes an outer housing 12 and a cone 14 that is integrally formed with a cone load cell 16 and a sleeve load cell 18. Seals 20 are disposed between the housing 12 and the cone load cell 16. The cone 14 is used to penetrate into the soil and the load is measured.

In FIG. 1B, a second example of a penetrometer 10′ having a cone 30 as a separate element from a cone load cell 32 and a sleeve load cell 34 is shown. An overload protection device 36 is disposed between the cone load cell 32 and the sleeve load cell 34. An outer housing 37 has threads 38 that secure the housing 37 to the sleeve load cell 34. Seals 40 are used to seal the housing 37 to the load cells 32, 34.

In FIG. 1C, a third example of a cone penetrometer 10″ is illustrated. In this example, a cone load cell 50 has a cone 52 and a cone load cell 54 that are integrally formed as a single unit. A sleeve load cell 56 is directly adjacent to the load cell 54. A housing 58 is coupled to the sleeve load cell 56 and the cone load cell 54. Seals 60 are disposed between the housing 58 and the cone 52.

In the present system, a controlled mechanical apparatus is used to penetrate the top layer of soils (1 to 3 inches) and record, in real time, with interaction properties such as force, deformation, movement, and flow. The system consists of a rigid wheel with a conic edge shape, mimicking the standard Dynamic Cone Penetration (DCP) test. The present system uses a DCP test characterizing soil shear deformations and strength using a moving disc instead of the typical stationary rigid cone. One challenge is replicating the accuracy of stationary testing methods with the dynamic apparatus, which has been accomplished in the apparatus described below

The form factor and attachment constraints were identified to equip the active measurement apparatus with force and deformation sensing modules. Integrated devices with active sensing and data sampling were designed, developed, and tested. The primary challenge in this step was extracting and cleaning the data to achieve meaningful correlations with standard soil characterization methods. The initial objective of data extraction at a 100 Hz sampling rate was met.

A simplified efficient model was developed to overcome the limitations of traditional soil models, which are typically based on finite element methods or other computationally intensive approaches. This model was designed for real-time sensing and computation during vehicle field operations, enabling effective integration with the sensing apparatus data.

Advanced numerical methods combining the Finite Element Method (FEM) and Discrete Element Method (DEM) were employed to validate experimental data and generate synthetic data for a wide range of cases beyond what could be achieved experimentally:

FEM was used to model the rigid wheel interacting with the soil. This method allowed for detailed analysis of the forces, stresses, and strains acting on the wheel under various conditions.

Specific objectives of the present system include: (1) Soil Properties Analysis: Variations in parameters such as cohesion, friction angle, and bulk modulus were analyzed to evaluate their influence on the interaction; (2) Strain Measurement: The strain experienced by the wheel was evaluated in response to changes in soil properties; (3) Deformation Behavior: The overall soil deformation and stress distribution were observed during wheel-soil interaction.

Dynamic Soil Characterization: Unlike traditional Cone Penetration Test (CPT) methods, this invention enables real-time, dynamic soil classification while a vehicle is in motion.

Integrated Sensor Wheel: The system features a rigid wheel sensor array that actively measures shear strength, deformation, and bearing capacity in real-world conditions.

Real-Time Computational Modeling: Combines FEM, DEM, and neural networks to process sensor data on-the-fly for autonomous vehicle control.

Autonomous Terrain Adaptation: First system to directly link real-time soil response to vehicle maneuverability adjustments, improving off-road AV stability in varying terrains.

Multi-Platform Integration: Designed for integration into various platforms, including ground vehicles, autonomous off-road systems, and UGVs (Unmanned Ground Vehicles)

Discrete Element Method (DEM): DEM was applied to model the granular behavior of the soil, providing insights into particle-level interactions and deformation under dynamic loading conditions.

Specific objectives of the present system include: (1) Parameter Sensitivity: The impact of soil particle interaction parameters (e.g., particle size, shape, and stiffness) on the results was analyzed; (2) Large Deformation Study: Fluid-like behavior and large-scale deformations of soil during the cutting process were captured; (3) Strain Response: The strain experienced by the soil particles during interaction with the rigid wheel was assessed, providing a more detailed understanding of the mechanics of soil behavior.

The combined FEM-DEM approach bridged the gap between rigid wheel analysis and the complex, granular nature of soil, ensuring a comprehensive understanding of the wheel-soil interaction. This dual-method strategy not only validated experimental results but also enabled the generation of synthetic data to cover a broad range of soil conditions and scenarios.

The complete system was integrated and tested in both laboratory settings and vehicle-based trials. These tests demonstrated the system's capability to measure and analyze soil properties in real-world conditions, ensuring its readiness for practical application.

The Cone Penetration Test (CPT) has become a cornerstone in geotechnical engineering for analyzing and characterizing soil properties. Initially developed for lunar soil analysis, the CPT was later adapted for terrestrial applications to classify and evaluate soils with precision, particularly in clay and soft soil regions, to ensure the safety of construction projects. Today, the CPT is widely employed by geotechnical engineers in the design and assessment of buildings, roads, bridges, and other critical infrastructure.

The CPT involves driving a cone-shaped penetrometer into the ground at a constant rate using a hydraulic machine. The penetrometer is equipped with load cells to measure vertical and horizontal forces, capturing key resistance parameters as the cone penetrates the soil. The primary objective of the test is to quantify the resistance encountered by the penetrometer, with measurements including cone or tip resistance (derived from the strain sensor), sleeve friction, and pore water pressure. These parameters are subsequently used to predict essential geotechnical properties such as soil bearing capacity, settlement potential, and soil behavior classifications.

The significance of the Cone Penetration Test (CPT) in this disclosure lies in its ability to utilize multiple data points to predict soil classification accurately through systematic analysis and test comparisons. In geotechnical engineering, two key parameters measured during the CPT are the cone or tip resistance (F_{Tip}) and the sleeve friction (S_{F}). These parameters are combined to calculate a normalized friction ratio, expressed as:

$${F}_{r} = \frac{{S}_{F}}{{F}_{Tip}}$$

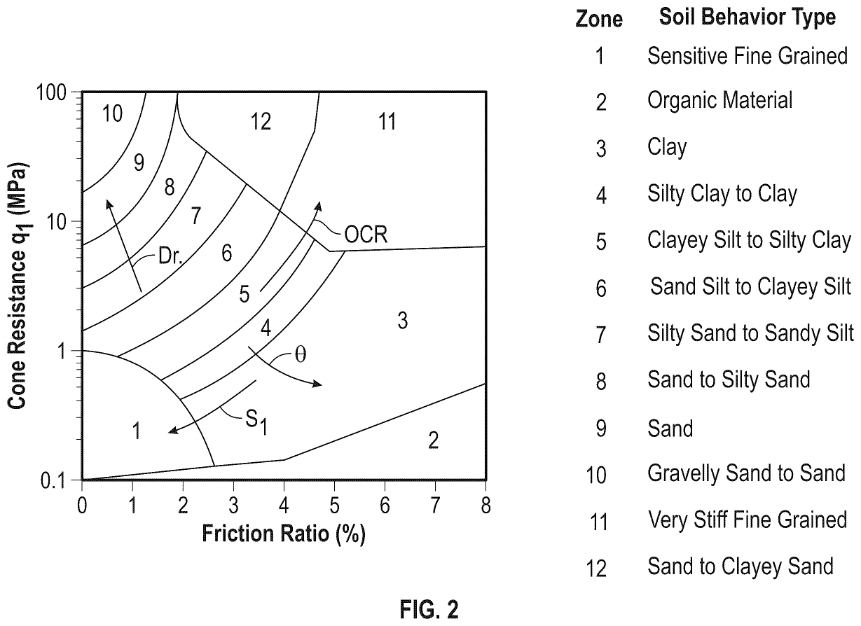

Referring now to FIG. 2, the Cone Penetration Test (CPT) computes this friction ratio in real time and compares it with the recorded cone or tip resistance to classify soil types with high precision. By performing multiple trials, the relationship between tip resistance and friction ratio can be visualized through a graph as shown in FIG. 2 providing an effective tool for soil classification and behavior analysis.

The present system simulates the principles of the stationary or static Cone Penetration Test (CPT) using dynamic soil reactions, where terrain properties continuously evolve under moving loads, unlike the static conditions assessed in traditional CPT applications. As a result, static CPT data cannot be directly applied to this study. Instead, the core principle of the CPT, which relies on resistance and friction measurements, is adapted to evaluate soil behavior in dynamic conditions.

The present system replicates the graphical representation produced by CPT tests, but instead of using static penetration (tip) resistance, the data is derived from dynamic soil interactions. This includes measuring the resistance encountered by the wheel tip as it moves through the terrain in real time, along with the forces required for traversal. These forces involve a combination of friction and cohesion, both critical factors in understanding terrain response to dynamic loads. By employing this modified methodology, a comprehensive classification system for soil behavior under varying dynamic conditions has been developed.

Another aspect of the disclosure is the determination of the bearing capacity of the terrain over which the vehicle operates. This information is used for autonomous vehicles to make real-time maneuvering decisions based on soil strength and stability. In geotechnical engineering, the most widely used equation for estimating soil bearing capacity under static loading conditions is Terzaghi's Bearing Capacity Equation:

qᵤ=c×N_{c}+γD_{f}N_{q}+0.5γBN₆₅ (1)

In this equation 1, c represents cohesion, γ is the unit weight of the soil, and D_{f} and B are structural parameters related to foundation geometry. The terms Nc, Nq, and Nγ are bearing capacity factors. Terzaghi's equation applies to static structures, where the load remains constant, such as building foundations. However, since this research focuses on dynamic loading conditions, where a moving wheel continuously interacts with the soil, modifications are required to account for these interactions.

For dynamic systems, such as a wheel traversing deformable terrain, Bekker's Equation for off-road vehicles provides a more suitable model. This equation describes the relationship between the vertical pressure exerted by a moving wheel and the resulting soil deformation, allowing for a dynamic assessment of terrain strength:

$${q}_{dyn} = {k}_{c} \times \frac{z}{b} + {k}_{ϕ} \times {( \frac{z}{b} )}^{n}$$

where z represents sinkage depth, or the depth of soil deformation caused by the wheel, b is the contact width of the wheel, kc is the modulus for cohesive soils, kφ is the modulus for frictional soils, n is the soil deformation exponent, typically ranging between 0.5 and 1.5, depending on soil type. This equation forms the basis for the second objective of this research: determining soil strength and bearing capacity after classification. Once the soil type is identified using a modified version of the Cone Penetration Test (CPT) (refer to FIG. 2), the parameters required for Bekker's equation can be estimated. Understanding soil classification enables the approximation of cohesion modulus (kc), friction modulus (kφ), and soil deformation constant (n), providing a comprehensive measure of terrain strength.

This equation is fundamental to the second research objective. The first step involves soil classification using dynamic CPT principles, while the second step focuses on determining soil strength using Bekker's equation based on real-time sensor data. The following section details the design, which involves adapting the CPT concept into a rotating wheel for real-time soil classification.

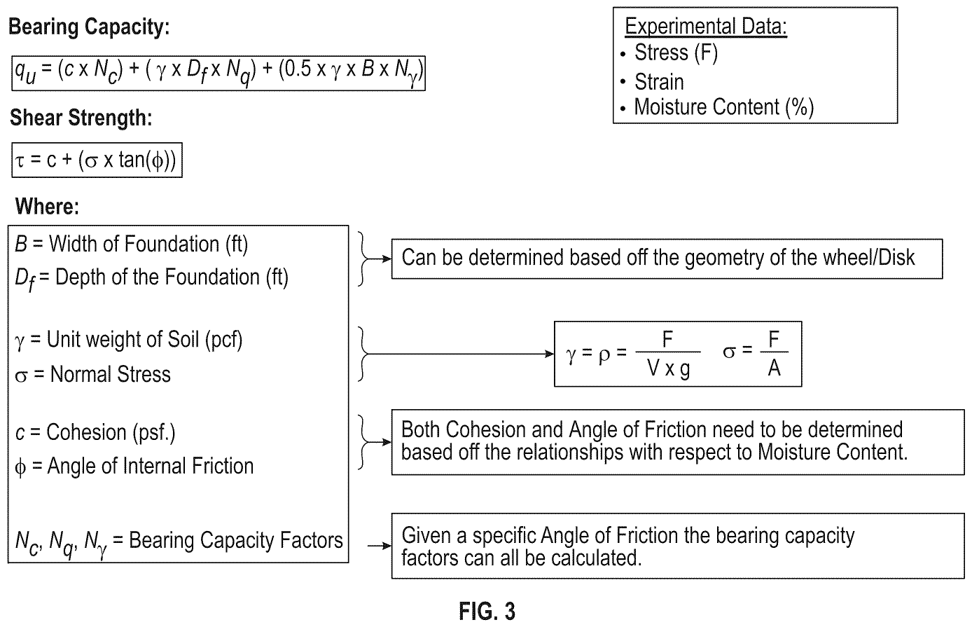

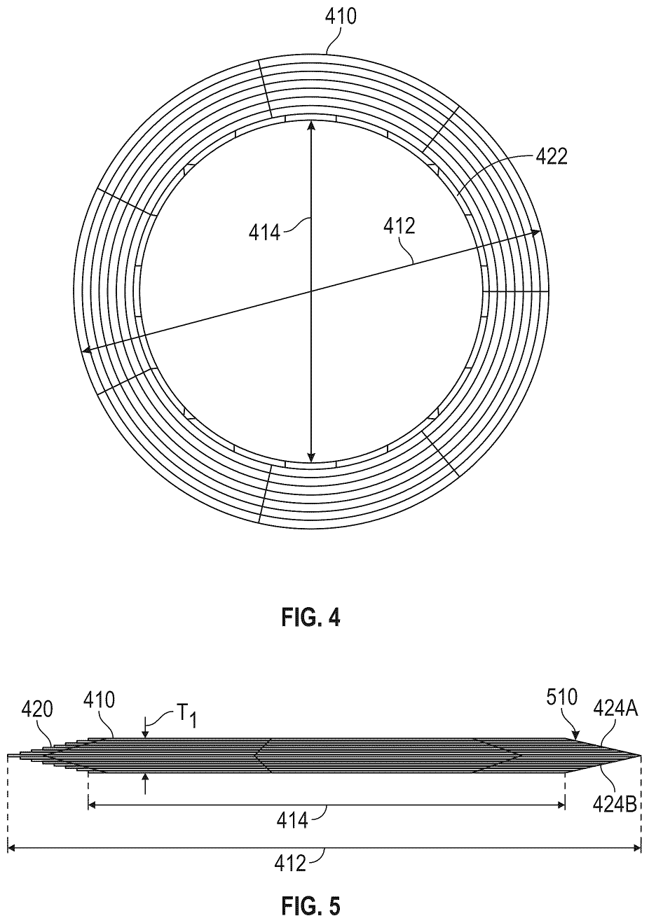

Referring now to FIG. 3, a summary of the formulas used for bearing capacity and sheer strength are set forth. The width of the foundation and depth of the foundation can be determined using the geometry of the wheel disk. The unit weight of soil and normal stress may also be easily determined based on the formulas set forth. The cohesion and angle of friction are determined based on the relationships with respect to the moisture content. The bearing capacity factors may be calculated from a specific angle of friction.

Referring now to FIGS. 4 and 5, a side view and edge view of a wheel 410 is illustrated. The wheel 410 has an outer diameter 412, an inner diameter 414 and has an overall thickness T1 of about 1 inch. The difference between the outer diameter 414 and the inner diameter 412 form a cone or point 420 that extends around the circumference of the wheel. In the present example, the outer diameter is about 6.7 inches. An inner lip depression of about 0.03125 inches at 422 is illustrated. The material thickness of the edge point 420 is about 0.0625 inches in the constructed example. An angle 510 of the edge of the wheel is formed from two walls 424A and 424B that form an angled surface.

When designing the sensor system, the goal was to replicate the soil interaction mechanics of the Cone Penetration Test (CPT) while adapting them for dynamic conditions. In a traditional CPT, the penetrometer penetrates the soil vertically at a controlled angle and speed, driven by hydraulic pressure. In contrast, the proposed design utilizes a rotating wheel that interacts with the soil horizontally at a predefined angle and speed, propelled by vehicle motion rather than a hydraulic system.

To maintain functional similarity with the CPT, the cross-sectional profile of the wheel mirrors the cone penetrometer design (as shown in FIG. 1). However, instead of a stationary penetration test, the cone undergoes continuous 360-degree rotation, forming a wheel-shaped sensor. This adaptation allows the system to continuously interact with the terrain, dynamically measuring soil or tip resistance and behavior in real time.

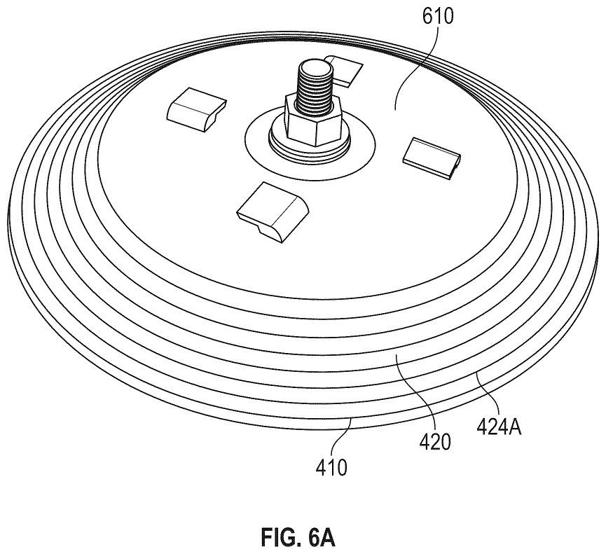

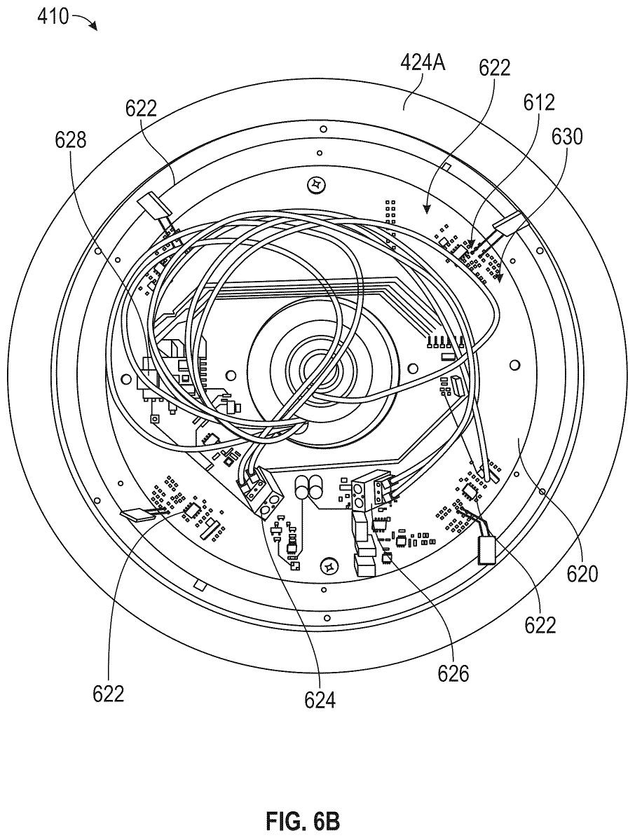

Referring now to FIGS. 6A and 6B, the wheel 410 is illustrated in further detail. FIG. 6A has side view of the wheel with a cover 610 thereon. In FIG. 6B the cover 610 is removed to reveal a hollow interior 612 which is completely hollow, while the outer layer is extremely thin. The design choice corresponds to the specific type of data being measured.

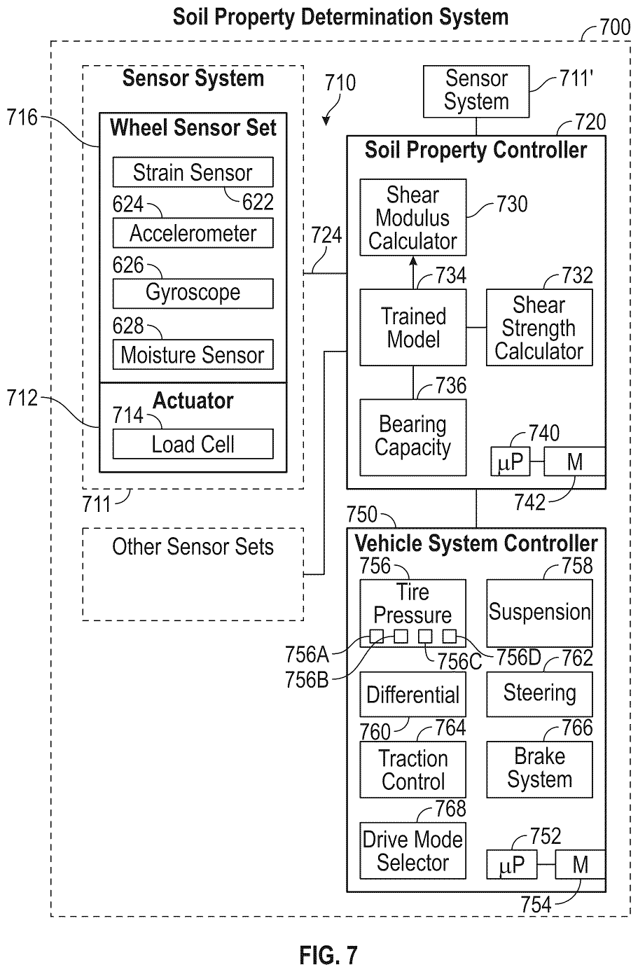

Referring also to FIG. 7, a vehicle 700 may include a soil property determination system 710 that has a circuit board 620 disposed within the hollow interior 612 and a sensor system 711 associated therewith. As illustrated and described below, a second rotating potentiometer may be associated with the vehicle 700 so that the soil bearing capacity and shear strength may be determined for each side of the vehicle as indicated by the second sensor set 711′, the details of which are not provided for simplicity because it duplicates sensor set 711. A wheel sensor set 716 is disposed on the circuit board 620 which is part of a soil property determination system 710. Strain sensors 622 are embedded inside the wheel 410 at the tip-edge on the circuit board 620 enabling the measurement of strain experienced by the wheel 410 during its cutting interaction with the soil. Additionally, the wheel 410 is mounted on a vertical actuator 712 with a displacement-fixed configuration, allowing for the measurement of reactionary forces exerted by the soil normal to the wheel using a load cell sensor 714 (or simply load cell). The load from the load cell 714 is used to determine stress. The system 710 adapts the Cone Penetration Test (CPT) principles for dynamic soil interactions at the surface level. Material selection was another factor in the design process. To ensure accurate strain and stress readings, the selected material needs to be durable enough to withstand environmental conditions while also being elastic enough to register measurable deformations. An initial prototype was fabricated using 3D-printed plastic, which served to validate the conceptual design and confirm that meaningful correlations between different soil types could be detected based on the collected data.

Testing of the plastic prototype yielded promising results, demonstrating that the design could effectively capture soil behavior. However, the plastic material deteriorated after repeated use, highlighting the need for a more durable solution. Aluminum was chosen for its weather resistance and mechanical properties, striking a balance between durability and the ability to accurately measure stress and strain.

The hollow wheel's outer sides were further reinforced with thin aluminum plates on both sides 424 and cut at a 30-degree angle, closely mimicking the geometry of the CPT penetrometer. This design ensures that the cutting action of the wheel closely parallels CPT behavior, while maintaining the structural integrity required for repeated dynamic testing.

The complexity of this task stems from the highly dynamic nature of the environment, where conditions change frequently and unpredictably. In static tests, such as those used in traditional CPT applications, the penetrometer typically reaches a maximum depth of 150 feet before the cone tip angle compromises the test's effectiveness. However, in this study, a moving vehicle allows for significantly longer testing distances in a single experiment. This introduces the challenge of accounting for constantly changing environmental factors, including soil composition, weather conditions, and terrain slopes. These variables create multiple research avenues and necessitate careful consideration to ensure the collection of meaningful and accurate data.

To address this complexity, the sensor system 716 integrates various sensors and gauges to provide reliable, multi-faceted data collection. The internal compartment 630 of the wheel 410 is equipped with the custom circuit board 620 featuring the four strain sensors 622, positioned at four equally spaced locations within the wheel 410, as previously described. The system 716 has the capacity to accommodate up to 16 strain sensors, allowing for higher data resolution. Additionally, the system incorporates an accelerometer 624 and a MEMS gyroscope 626 to track movement and orientation. A moisture sensor 628 is used to measure variations in soil water content. The sensor system 716 and the sensors 622-628, 714 collectively help identify and mitigate outliers or inconsistencies in large datasets, ensuring the integrity of the collected data.

The data from all sensors 622-628, 714 are transferred logged and communicated to a soil property controller 720 at a sampling rate of 400 Hz. This may be performed through a connection 724 wirelessly or through a wire. While this rate exceeds the minimum required for analysis, it ensures cleaner data and provides the necessary resolution to capture moment-to-moment variations during testing.

The soil property controller 720 has a shear modulus calculator 730 that determines a shear modulus for the soil based upon the sensors within the sensor system 711. A shear strength calculator 732 determines the shear strength of the soil also based upon the sensors within the sensor systems 711. The shear strength calculator 732 may be a trained classifier. Details of the shear modulus and the shear strength determinations are provided below. Both the shear modulus and the shear strength determined by the respective shear modulus calculator 730 and the shear strength calculator 732 are communicated to a trained model 734. The train model 734 may be neural network or another type of machine learning that uses the shear modulus and shear strength to determine a bearing capacity 736. The soil property controller 720 has a microprocessor and a memory 742. The microprocessor may be referred to as a processor. The memory 742 may be non-transitory computer-readable medium that includes instructions that are executable by the processor or microprocessor 740.

While the system is described as incorporated into the vehicle 700, other types of systems may include the sensor system 711 and the soil property controller. In a vehicle, a vehicle system controller 750 may receive the bearing capacity 736 so that various systems within the vehicle may be controlled to achieve maneuverability and stability of the vehicle 700. The vehicle 700 may be an operator controlled vehicle or may be an autonomous vehicle.

The vehicle system controller 750 may include a microprocessor 752 and a memory 754. The memory may be a non-transitory, computer readable medium that includes instructions that are executable by the processor for controlling the vehicle. The vehicle system controller may include various controllable elements that are controlled based upon the bearing capacity determined by the soil property controller 72. These include, but are not limited to, a tire pressure system 756 having tire inflation/deflation systems 756A-756D, a suspension system 758, a differential system 760, a steering system 762, a traction control system 764, a brake system 766 and a drive mode selector system 768. One or more of the systems 756-768 may be used in controlling the operation of the vehicle to achieve stability and maneuverability.

The tire pressure system 756 may control the tire pressure for one or more of the tires of the vehicle to provide the desired maneuverability. For example, in low traction situations, such as on sand, the tire pressures may be lower. This may performed with an inflator or deflator that is on the vehicle. For example, when entering a sandy soil condition, the tires may be deflated and after the sandy condition, the tires of the vehicle may inflated.

The suspension system 758 may also be controlled. Ride height of the vehicle as the position of the body of the vehicle relative to the wheels and suspension may be changed based on the various conditions.

The differential system 760 may be changed. This is particularly useful when one side of the vehicle is on a different type of soil having different bearing capacities.

The steering system 762 may be changed based on the bearing capacity of the soil in which it is driven. In loose soil, the steering control of the vehicle may be changed.

Control of the traction control system 764 may also be altered based upon the bearing capacity. By knowing the bearing capacity of the soil on different sides of the vehicle, the spin or rotation of the wheels may be altered differently on either side of the vehicle.

The brake system 766 may also be modified based upon the bearing capacity 736. Braking may be modified, in particular, when the bearing capacity of the soil is different at different locations of the vehicle, such as on either side.

A drive mode 768 may also be changed by the drive mode system 768. Different types of drive modes, such as snow, dirt, sand and hard surfaces, may alter the handling characteristics of the vehicle.

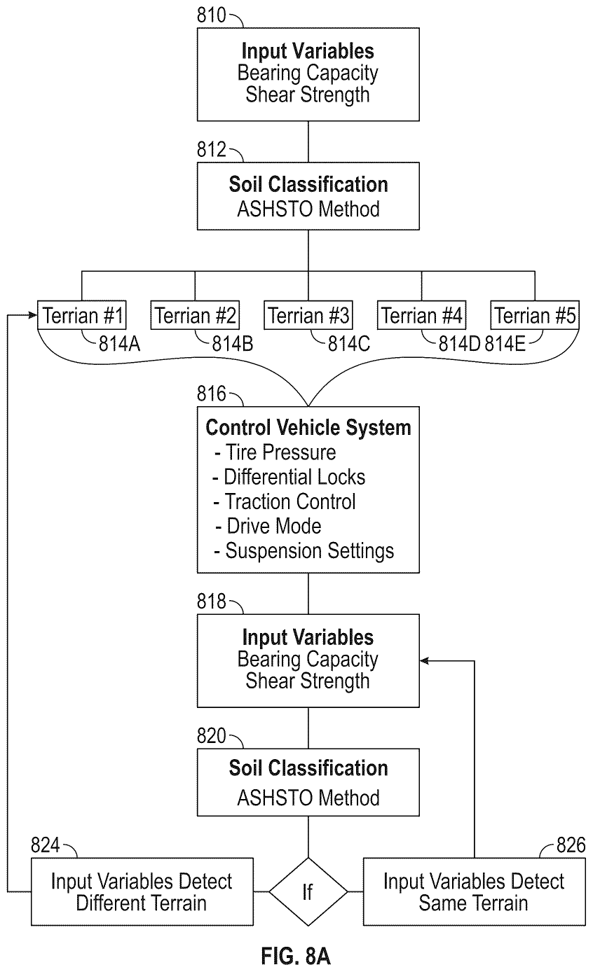

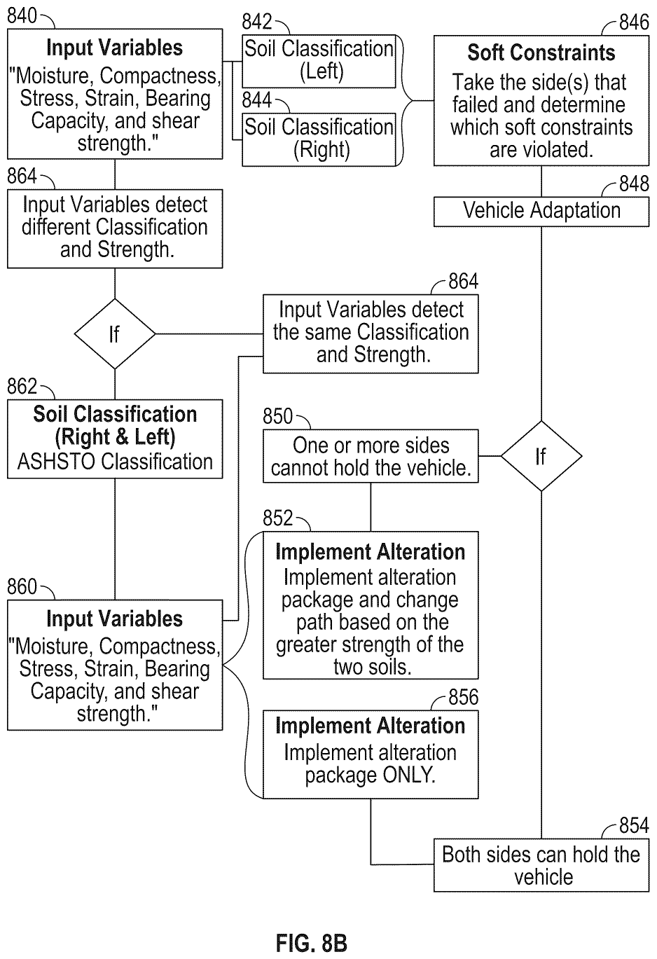

Referring now to FIG. 8A, a method for operating a vehicle is set forth. In step 810, input variables are determined. The input variables, such as bearing capacity and shear strength, may be determined from the sensor system 711 illustrated in FIG. 7. Details of the derivations of the input variables are described in greater detail below.

In step 812, a soil classification is performed. The soil classification may be performed according to an American Association of State Highway and Transportation Officials (ASHSTO) method. The ASHSTO method is based on stress and strain ratio charts such as that illustrated in FIG. 2 above, they are fixed and based off moisture and compactness. Based upon the soil classification, various types of terrains are determined in steps 814A-814E. Based upon the terrain, the various vehicle systems may be controlled in step 816. For example, the tire pressure system may automatically deflate or reinflate tires to provide the best traction balance between traction and tire protection, the differential may engage or disengage based upon the need for synchronized wheel rotations which may be important in low traction environments, the traction control system may prevent wheel spin in slippery conditions or allow more slip in rocky conditions. The drive mode, such as four-wheel drive and all-wheel drive, may be changed by allowing lower gear ratios in four-wheel drive so that more torque at lower speeds is provided to the wheels when navigation is within partially challenging terrains. The suspension settings of the vehicle may also be changed to increase ground clearance for rocky terrain or lower the height of the vehicle for high speed travel on flat surfaces.

After step 816, the system repeats in terms of determining input variables again. The input variables are constantly monitored and, in step 820, the soil classification method is again determined. If the input variables detect different terrain in step 824, the system may change the control of the vehicle system in step 816. If the input variables detect the same terrain in step 826, the input variables in step 818 are again performed.

Referring now to FIG. 8B, the flowchart illustrated in FIG. 8A presumes all of the wheels are on the same type of soil conditions. In most circumstances, this will be true. However, the system may provide two different sets of sensors for determining the soil capacity on both sides of the vehicle and may be controlled differently. In FIG. 8B, input variables are obtained in step 840. The input variables may include moisture, compactness, stress, strain, bearing capacity and shear strength. The input variables may be derived from the sensor system 711 described above. In step 842, the soil classifications may be determined using the ASHSTO classification systems in steps 842 and 844. Step 842 determines the soil classification for the left side of the vehicle while step 844 is for the right. In step 846, the soft constraints may be taken into consideration. The soft constraints may be used to determine which soft constraints are violated and which side of the vehicle has failed or changed. In step 848, the vehicle adaptions may be performed to alter the operation of the vehicle. As mentioned above, the tire pressure, differential locks, traction control, drive mode and suspension settings may all be altered. In this example, however, the control of each side of the vehicle may be performed independently or controlled together to obtain the vehicle stability or maneuverability desired. In step 850, if one or more sides cannot hold the vehicle, an alternation package may be implemented that changes the path of the vehicle based on the greater strength of the two different types of coil sensed. That is, the better soil may be chosen as the path and the vehicle moved in that direction using the steering system, for example. In step 848, when both sides of the vehicle can be held, step 856 implements only an alteration package without changing the path. After steps 852 and 856, step 860 again determines input variables in a similar manner to that set above with respect to step 840. In step 862, the right and left classifications may be determined and step 864 is executed when input variables detect the same classification and strength for the soil. After step 864, step 860 is again performed. In step 864, when the input variables detect different strengths or classifications in step 864, step 860 is performed.

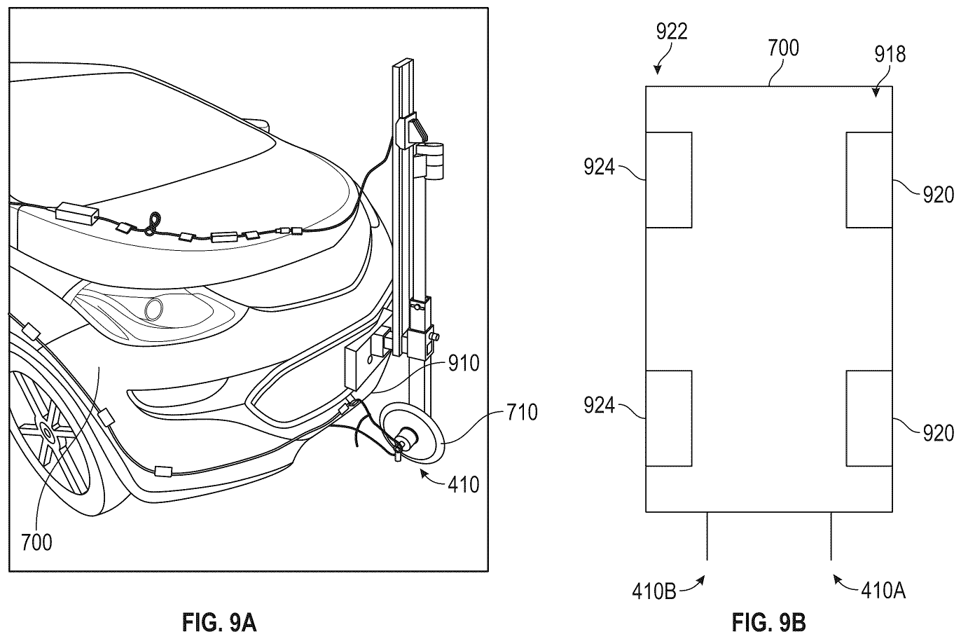

Referring now to FIG. 9A, the wheel 410 and the completed sensor system 710 are designed for integration into the vehicle 700. The attachment mechanism 910 is modeled after a standard vehicle hitch, similar to those used for towing trailers and other equipment. However, in this design, the hitch is modified to attach to the front of the vehicle 700. A custom-built mounting system secures the wheel to the hitch, ensuring stability during operation. The entire system is powered internally from the vehicle, making it self-sufficient throughout testing.

In FIG. 9B the vehicle 700 has a left side with wheels 920 and a right side 922 have wheels 924. The sides 918 and 922 respective penetrometer wheels 918 and 922. The penetrometer wheels have the sensor sets described above. As mentioned above the two sides of the vehicle 700 may have various control systems that may be controlled independently based on the bearing capacity of the soil. This may be particularly useful for autonomous vehicles.

### Experimental Testing

The experimental design was evaluated on three distinct soil types: clay-silt, sand-gravel, and organic material. These soil types were selected based on their diverse physical properties and prevalence in natural environments. Testing across such a varied range of soils provides a robust assessment of whether the research objective—real-time soil classification based on strain response—can be effectively achieved.

The device was tested in a controlled environment to minimize external variability and maintain consistent testing parameters. Each soil type was evaluated under identical conditions, with constant speed and moisture content, ensuring a reliable baseline strain response for each soil type. Data plots were collected as frequency-based measurements, as the strain gauge registers zero (or near zero) when not in contact with the soil. The key metric of interest was the peak strain values, which correspond to the maximum deformation experienced by the gauge upon interaction with the soil. This typically occurs when the gauge is positioned vertically against the soil, providing a direct measurement of the true strain response, which is critical for soil classification.

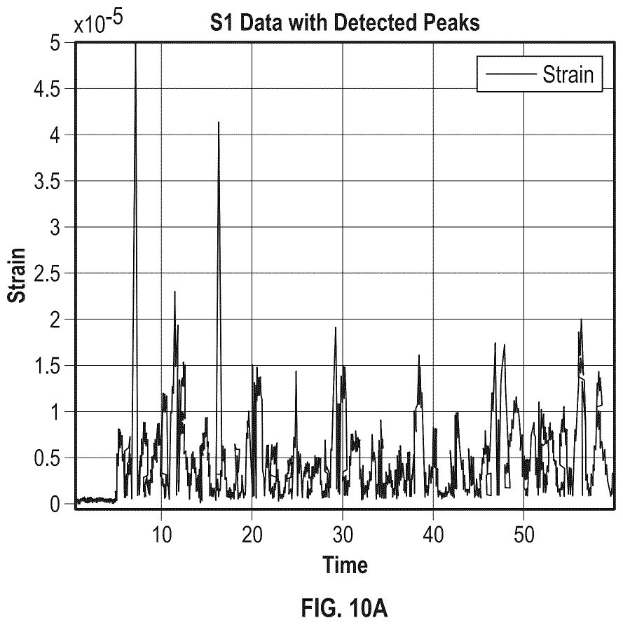

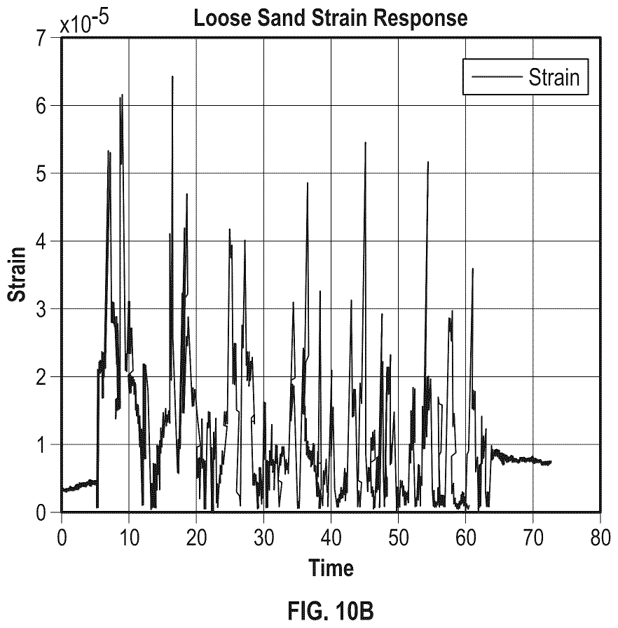

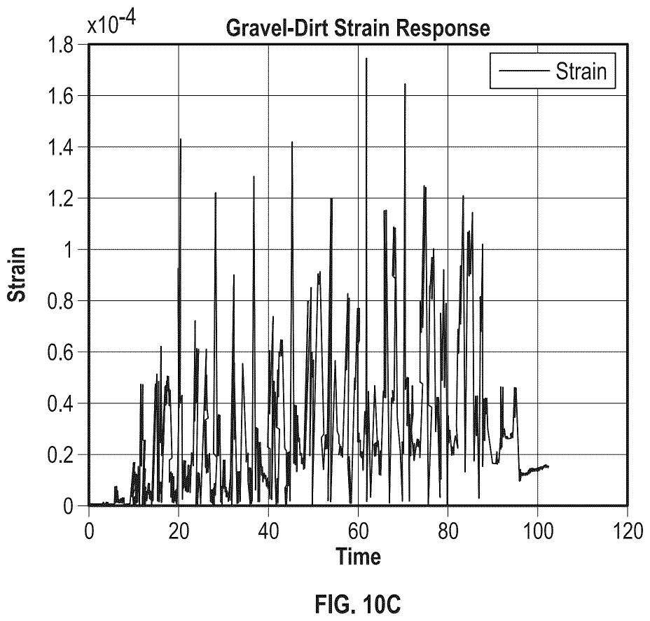

Referring now to FIG. 10A, the first test was conducted on organic soil, which is primarily composed of decomposed plant material and organic matter. Organic soil is characterized by its soft, loose structure, high air content, and significant water retention when wet. Due to these properties, the average strain response recorded during testing was 1.894×10⁻⁵. The relatively low strain response aligns with the soil's non-compactable and highly porous nature. The strain response plot for organic soil is presented in FIG. 10A.

Referring now to FIG. 10B, the second test was conducted on loose, non-compacted sand, composed primarily of quartz mineral particles with particle sizes ranging from 0.06 mm to 2 mm. The strain response recorded for this soil type was 4.072×10⁻⁵, which was significantly higher than that of the organic soil. This increase in strain response is attributed to the denser particle arrangement and greater interparticle friction present in sand compared to the loosely structured organic soil. The strain response plot for the sand test is presented in FIG. 10B.

When comparing the results from organic soil and sand under identical conditions, the data clearly indicates a significant difference in strain response. The loose sand exhibited a 73% higher strain response than the organic soil, which can be attributed to its greater density and higher frictional resistance. This marked difference suggests that strain response serves as a reliable metric for distinguishing between softer, organic soils and denser, frictional soils under controlled conditions.

Referring now to FIG. 10C, the third test was conducted on a poorly graded gravel mixture containing silt and sand to evaluate how a composite material influences strain response. The larger gravel particles in the mixture contributed to increased soil strength and higher frictional resistance compared to loose sand alone. The average strain response recorded for this soil type was 9.708×10⁻⁵, which was significantly higher than both the organic soil and sand samples. This result aligns with expectations, as the interlocking gravel particles and their rough surface texture provide greater resistance to deformation. The strain response plot for this test is illustrated in FIG. 10C.

The findings of this study represent a significant advancement in the ability to classify soils using strain response measurements, successfully achieving the first objective of this research. By systematically testing three distinct soil types-organic material, sand, and a gravel-sand-silt mixture under controlled conditions, a consistent pattern in strain behavior was observed.

Strain responses below 2×10⁻⁵ were indicative of organic soils, while values ranging between 3×10⁻⁵ and 5×10⁻⁵ were associated with sands and sand-silt mixtures, reflecting their higher density and frictional properties. The highest strain responses, exceeding 9×10⁻⁵, were recorded for gravel mixtures, aligning with expectations that larger particles exhibit greater mechanical resistance.

These results confirm that strain data can reliably differentiate soil types, paving the way for real-time soil assessment in autonomous vehicle navigation. The next phase of this research focuses on utilizing Bekker's Equation to translate strain data into meaningful soil strength metrics, enabling more precise terrain prediction and improved decision-making in dynamic environments.

### Numerical Simulations

The objective of this study is to model the interaction between a cutting disk and soil, with a particular focus on the applied strain on the disk and the deformation behavior of the soil. The research aims to examine how variations in soil properties influence mechanical responses and to develop a computational framework for accurately predicting strain and deformation characteristics in different soil conditions.

One aspect of this study involves analyzing soil parameter variations, particularly the effects of cohesion, friction angle, and bulk modulus on disk strain and soil deformation. By systematically altering these parameters, the study seeks to understand how changes in soil composition and mechanical properties affect the tip resistance experienced by the cutting disk.

To assess soil deformation, the study investigates soil displacement and stress distribution under different loading conditions. By observing how different soil types behave when subjected to mechanical forces, valuable insights can be gained into terrain response and material failure mechanisms.

The Finite Element Method (FEM) is employed to simulate the mechanical interaction between the disk and soil. This approach allows for a detailed analysis of:
Soil Properties Analysis, where variations in cohesion, friction angle, and bulk modulus are examined to determine their impact on the cutting interaction.
Strain Measurement, which evaluates the stress-strain response of the disk as it moves through the soil, providing a quantitative measure of mechanical or tip resistance.
Deformation Behavior, where the study captures how soil reacts under different stress conditions and how these deformations influence the forces exerted on the disk.

In addition to FEM, Smoothed Particle Hydrodynamics (SPH) and Discrete Element Method (DEM) are utilized to model soil behavior with greater accuracy. These methods allow for a more detailed examination of:
Parameter Sensitivity, where the influence of particle interactions on overall soil behavior is analyzed, focusing on how variations in particle size, shape, and stiffness impact results.
Large Deformation Study, which captures fluid-like soil behavior and extensive deformation patterns as the disk cuts through the material.
Strain Response, where the mechanical reaction of the soil and disk is evaluated during the cutting process, providing insights into the fundamental mechanics of terrain interaction.

### Soil Behavior Simulation Techniques

### SPH (Smoothed Particle Hydrodynamics)

Smoothed Particle Hydrodynamics (SPH) is a mesh-free, particle-based computational method used to simulate the behavior of fluids and deformable solids. In this approach, each particle represents a mass of material with assigned properties such as density, velocity, and internal energy. Interactions between particles are smoothed over a defined distance, allowing for the natural handling of complex, free-surface flows. The SPH method is based on interpolation theory, where field variables at any point within the domain are approximated by summing contributions from a set of neighboring particles. This unique formulation enables SPH to efficiently model large deformations and free-surface interactions, making it a valuable tool for terrain and soil behavior simulations.

One of the primary advantages of SPH is its high adaptability, as it effectively handles large deformations and irregular particle distributions, making it well-suited for complex simulations. Additionally, SPH features a simplified neighbor-particle searching algorithm, which contrasts with traditional grid-based methods and allows for straightforward implementation. Another key benefit is that SPH employs explicit solutions, meaning it directly solves governing equations without requiring additional solvers. This makes the method computationally efficient for certain applications. Furthermore, SPH is highly compatible with parallel computing, as each particle calculation is independent, enabling the development of efficient parallel algorithms that improve processing speeds.

Despite these advantages, SPH has some limitations that must be considered. One key drawback is its reduced accuracy in small or finite deformation problems, where it tends to exhibit lower-order accuracy compared to traditional grid-based methods. Another challenge is instability, as SPH is prone to numerical oscillations and tensile instability, which can reduce the reliability of simulations. Additionally, computational efficiency remains a concern, as kernel functions and neighbor searches must be updated at each timestep, increasing computational costs, particularly for large-scale simulations.

While SPH presents certain challenges, its ability to model highly dynamic and deformable materials makes it an effective tool for soil simulation and terrain interaction modeling. Its combination with other numerical methods, such as the Discrete Element Method (DEM), allows for a more comprehensive representation of soil mechanics and cutting interactions in complex environments.

### DEM (Discrete Element Method)

DEM is a numerical technique used to simulate the behavior of granular materials and rigid body interactions. In DEM, materials are treated as a collection of discrete particles, each interacting through contact forces governed by contact models. Each particle is assigned physical properties, including mass, position, velocity, and rotation, while interactions are determined based on collision mechanics and force transmission. This method enables the modeling of particle flow, compaction, and the behavior of particulate systems under various loading conditions.

One of the primary advantages of DEM is its high adaptability, making it an effective tool for modeling granular flows and capturing complex particle interactions. This capability makes DEM particularly suitable for simulations involving bulk solids, granular materials, and particulate transport systems. Another strength of DEM is its ability to provide detailed information at the granular level, offering insights into force chains, particle movement, and how material behavior evolves under stress. Similar to Smoothed Particle Hydrodynamics (SPH), DEM employs explicit solutions, directly solving governing equations for particle interactions and motion at each time step. Additionally, DEM is highly compatible with parallel computing, as particle interactions are computed independently, allowing simulations to scale efficiently on high-performance computing (HPC) systems.

Despite these advantages, DEM has some limitations. One significant challenge is computational efficiency, as DEM requires constant neighbor searching and interaction calculations at every timestep. This computational demand becomes especially costly when dealing with large-scale simulations involving thousands to millions of particles. Another limitation is that DEM does not directly calculate stress-strain relationships, unlike continuum-based methods such as Finite Element Modeling (FEM). Instead, it calculates particle forces and movement, which must be post-processed to infer bulk material behavior. Additionally, parameter calibration in DEM presents a challenge, as accurately defining friction, stiffness, and damping properties is essential for achieving realistic material behavior. Calibrating these parameters often requires extensive experimental validation and fine-tuning to ensure the accuracy of the model.

DEM remains an essential tool for simulating granular interactions, particularly in scenarios where particle-level analysis is necessary. By combining DEM with other numerical methods, such as SPH and FEM, a more comprehensive understanding of soil mechanics, cutting interactions, and terrain response can be achieved, enhancing the predictive capabilities of soil-vehicle interaction models.

| SPH | DEM |
|---|---|
| Lagrangian-based method | Lagrangian-based method |
| Continuum-based | Discrete |
| Stress-Strain are derived | Stress-Strain cannot be |
| directly | calculated directly |
| Computationally | Computationally |
| demanding due to | demanding due to |
| neighbor searching and | neighbor searching |
| interaction calculations. | and interaction |
| | calculations. |
| Can capture large | Can provide detailed |
| deformations | information about force |
| | chains and particle |
| | movement. |

### Simulation Methods

### HyperMesh

HyperMesh is a high-performance finite element pre-processor that offers advanced meshing and modeling capabilities for engineering simulations. It enables the creation of detailed models for structural, fluid, and thermal analysis, making it a widely used tool in computational mechanics. In this study, HyperMesh was utilized to create the wheel model, which was then converted into a flexbody for modal analysis. The Modal Superposition Method was applied, representing the deformation of the flexbody as a sum of mode shapes, where each mode corresponds to a specific vibration pattern. This approach significantly reduces computational complexity compared to performing a full Finite Element Analysis (FEM) while maintaining a low error margin. By focusing on modal contributions, this method efficiently handles large deformations and complex geometries without compromising accuracy.

### MotionSolve

MotionSolve is a multidisciplinary simulation software used for analyzing and optimizing the dynamic behavior of mechanical systems. It allows for the simulation of real-world motion, structural dynamics, and control interactions, making it particularly well-suited for applications such as vehicle dynamics, robotics, and machinery. In this research, MotionSolve was employed to simulate the dynamics of the vehicle and the additional wheel. The flexbody created in HyperMesh was imported into MotionSolve and integrated into the vehicle's dynamic simulation, allowing for an accurate representation of wheel deformation and its interaction with the vehicle under dynamic conditions. By utilizing modal analysis, the flexbody's behavior was efficiently modeled while maintaining a low error margin, improving the accuracy of the simulation without the computational burden of a full FEM.

### EDEM

EDEM is an advanced Discrete Element Method (DEM) software used for modeling and simulating bulk material behavior, particularly granular and particulate systems such as soil. It provides detailed insights into particle interactions, material deformation, and stress distribution, making it a critical tool for studying soil-vehicle interactions. In this study, EDEM was coupled with MotionSolve, enabling a comprehensive analysis of both vehicle dynamics and soil behavior. EDEM models the soil, capturing interactions between soil particles and dynamically adjusting in real-time according to the motion and forces exerted by the vehicle. This integration enhances the accuracy of the soil-vehicle interaction simulation, providing a realistic representation of terrain response under dynamic loading conditions.

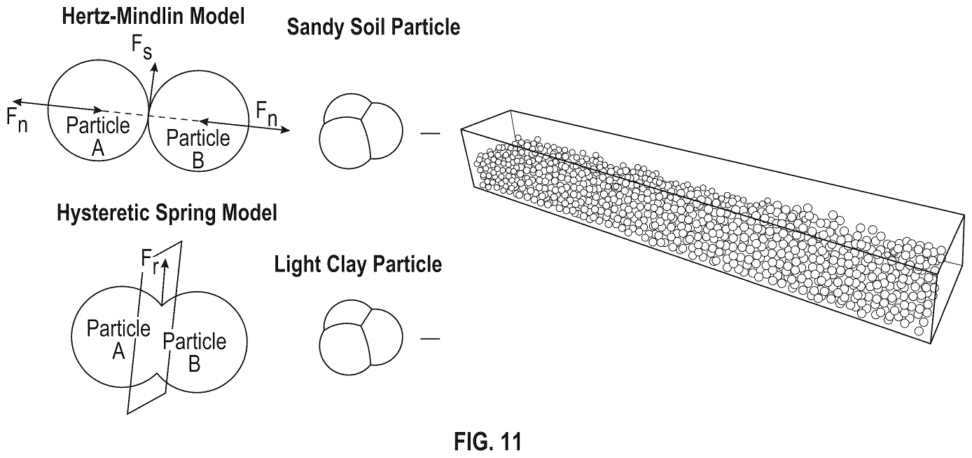

Referring now to FIG. 11, a representation of a physical soil model is set forth. The contact interactions between sand-loam particles (comprising sand and silt) are modeled using the Hertz-Mindlin (no slip) contact model, which considers only contact and friction forces, without any bonding mechanism between particles. This model is well-suited for granular materials where particle interactions are primarily governed by elastic deformation and frictional resistance at the contact points.

In contrast, clay particle interactions are represented using the hysteretic spring contact model, which accounts for adhesive forces between particles. In this model, particles stick together once a defined force threshold is exceeded, mimicking the cohesive nature of clay. Unlike the Hertz-Mindlin model, the hysteretic spring model does not allow for full recovery of particle separation, meaning that once the bonding force is broken, the particles remain disconnected, reflecting the irreversible behavior of clay aggregation in real-world conditions.

### Numerical Results:

To simulate the complete model, the soil simulation from EDEM is integrated into the dynamic simulation in MotionSolve, allowing for a coupled analysis of vehicle dynamics and soil behavior. Within this framework, EDEM calculates the total force acting on the wheel, along with key soil behavior metrics, including velocity, displacement, and the force exerted by soil particles. These parameters provide critical insights into how the wheel interacts with different soil types and how terrain properties influence vehicle motion.

To visualize the strain and stress distribution on the wheel, the output data from MotionSolve is processed and analyzed using HyperGraph. This step enables the generation of detailed plots that illustrate stress variations, deformation patterns, and mechanical responses under different operating conditions. By combining these simulation tools, a comprehensive representation of the soil-wheel interaction is achieved, ensuring accurate assessment and validation of the proposed model.

| | Shear | Shear | Shear | Total |
|---|---|---|---|---|
| | strain | strain | strain | force |
| Soil | z | zx | zy | Z |
| Sand | 3.3e−5 | 1.4e−5 | 2.9e−5 | 6150N |
| Clay | 2.6e−4 | 6.8e−5 | 5e−4 | 14400N |
| 33% sand, 45% | 2e−5 | 3e−6 | 1.9e−5 | 1300N |
| silt and 22% clay | | | | |
| 42% clay, | 6e−4 | 8.2e−5 | 6.4e−4 | 8400N |
| 47% silt, and | | | | |
| 10% sand | | | | |
| Sandy Loam | 8e−5 | 2.3e−5 | 2e−5 | 6844N |

### Bearing Capacity and Shear Strength Calculation

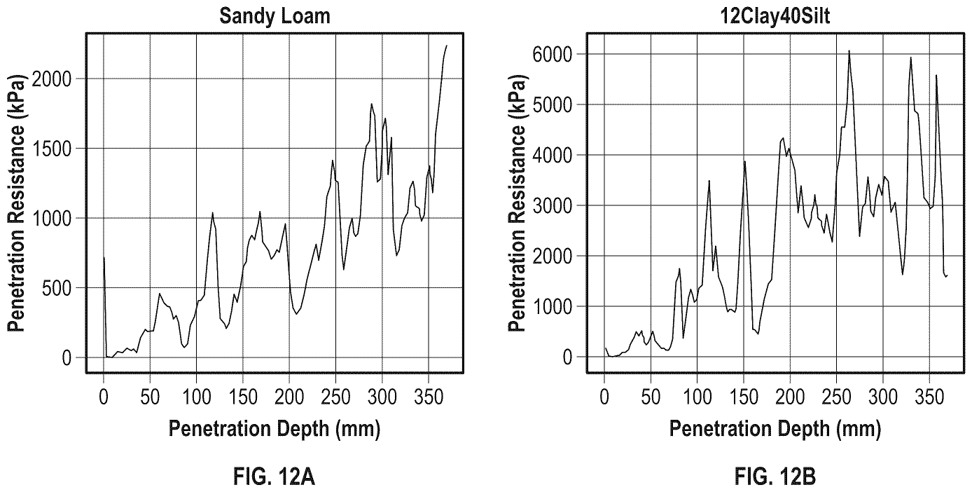

Referring now to FIG. 12. T bearing capacity and shear strength are parameters in soil modeling, yet they are not directly input into the simulation software. Instead, an additional simulation is required to determine these properties. To achieve this, a Cone Penetration Test (CPT) is simulated using EDEM, replicating the real-world procedure on the generated soil model.

The objective of this simulation is to measure penetration resistance as a function of penetration depth, mirroring the methodology used in experimental CPTs. By applying the same empirical formulae used in physical CPT tests, the bearing capacity and shear strength of the simulated soil can be calculated. This approach ensures that the soil properties in the numerical model are accurately characterized, allowing for a more realistic representation of soil behavior in further analyses.

### Correlation Between Measured Parameters and Soil Parameters:

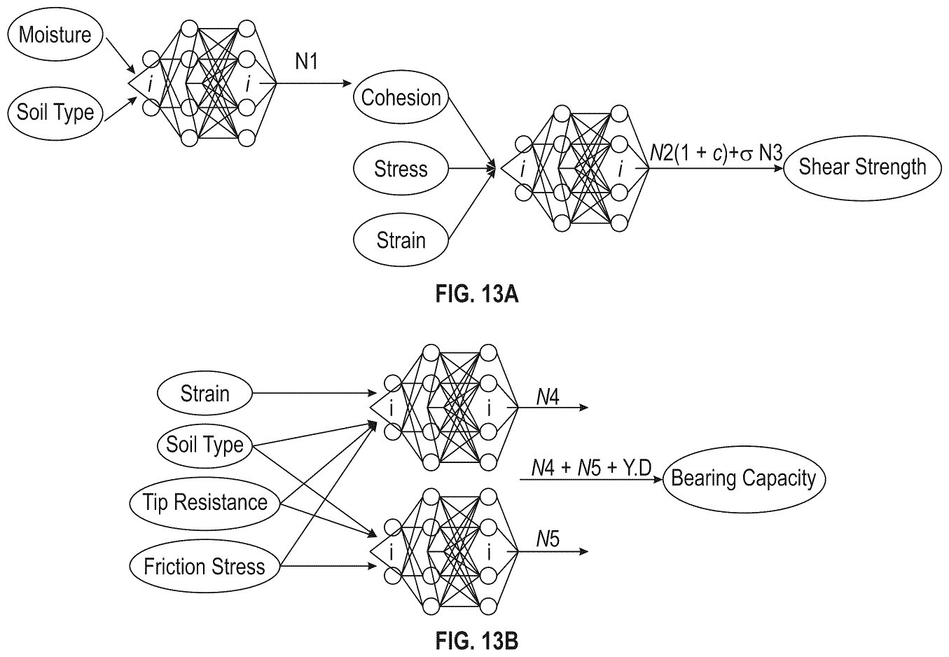

Referring now to FIGS. 13A and 13B, the objective is to establish a correlation between measurable physical quantities and the soil parameters required for vehicle control calibration. To achieve this, a mechanics-informed neural network is developed to identify the relationship between strain, stress, and key soil properties, including shear strength and bearing capacity. FIG. 13A used neural networks N1 and N2 to obtain shear strength. FIG. 13B uses neural networks N1 and N2 to obtain bearing capacity.

This approach leverages physics-based constraints to enhance the predictive capability of the neural network, ensuring that the learned relationships remain consistent with soil mechanics principles. By integrating real-time strain and stress data from sensor measurements, the model enables accurate estimation of soil properties, facilitating improved vehicle control strategies in response to varying terrain conditions.

In FIG. 13B, shear strength is defined as function of the cohesion c, effective normal stress σ and friction angle φ, t=c+σ tan(φ). N1 predicts the cohesion which is function of the moisture and depends on the soil type. N2 improves the prediction of the cohesion using the measured strain and stress data. N3 predicts tangent of the friction angle.

In FIG. 13B, bearing capacity is modeled in a similar way. It is defined as function of the tip resistance q_{c}, sleeve friction stress σ_{fs}, weight unit γ and depth D. q=N_{CPT}*ƒ(σ_{fs},D,q_{c})+γ. D. N4 here predicts the function ƒ. N5 improves the prediction of the second term using the measured strain and stress data.

In the foregoing description an additional sensing wheel is described. However, the sensors may be incorporated into the tires of a vehicle. Further the system could also be wired to the vehicle (rather than wireless) and use the power of the vehicle (rather than being self powered).

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python.

What is claimed is:

1. A rotating penetrometer for use in soil comprising:
an actuator arm;
a hollow wheel having an outer wall;
a circuit board disposed within the hollow wheel;
a plurality of strain sensors disposed on the circuit board generating strain signals;
an accelerometer disposed on the circuit board generating an acceleration signal;
a gyroscopic sensor generating an orientation signal;
a load cell coupled to the actuator arm; and
a moisture sensor.

2. The rotating penetrometer of claim 1 wherein the outer wall comprises aluminum.

3. The rotating penetrometer of claim 1 wherein the outer wall comprises an angled surface.

4. The rotating penetrometer of claim 3 wherein the angled surface comprises an angle between 25 and 35 degrees.

5. The rotating penetrometer of claim 1 wherein the plurality of strain sensors comprises four strain sensors.

6. The rotating penetrometer of claim 1 wherein the load cell determines a horizontal and vertical load on the actuator.

7. A soil property determination system comprising:
the penetrometer of claim 1;
a soil property controller receiving the signals and determining a bearing capacity of the soil.

8. The soil property determination system of claim 7 wherein the soil property controller determines a shear modulus and a shear strength to determine the bearing capacity.

9. The soil property determination system of claim 7 wherein the soil property controller determines a shear modulus and a shear strength to determine the bearing capacity based on at least one a trained model.

10. The soil property determination system of claim 9 wherein the bearing capacity of the soil is determined from a trained model in response to strain, soil type, tip resistance and friction stress.

11. The soil property determination system of claim 9 wherein the shear strength of the soil is determined from a trained model in response to moisture, soil type, a stress and a strain.

12. A vehicle comprising:
the soil property determination system of claim 7;
a vehicle system; and
a vehicle controller generating a control signal for controlling the vehicle system based on the bearing capacity of the soil.

13. The vehicle of claim 12 wherein the vehicle system comprises a tire pressure system inflating or deflating tires of the vehicle based on the bearing capacity.

14. The vehicle of claim 12 wherein the vehicle system comprises a differential engaging or disengaging based on the bearing capacity.

15. The vehicle of claim 12 wherein the vehicle system comprises a traction control system controlling the vehicle based on the bearing capacity.

16. The vehicle of claim 12 wherein the vehicle system comprises a drive mode selector selecting a drive mode based on the bearing capacity.

17. A vehicle comprising:
a first rotating penetrometer;
a second rotating penetrometer;
a soil property determination system coupled to the first rotating penetrometer and the second rotating potentiometer and determining a first bearing capacity and a first shear strength associated with the first rotating penetrometer, and a second bearing capacity and a second shear strength associated with the second rotating penetrometer;
a vehicle system;
a vehicle controller coupled to the soil property determination system and the vehicle system receiving the first bearing capacity, the first shear strength, the second bearing capacity and the second shear strength, said vehicle controller generating a control signal for controlling the vehicle system based on the first bearing capacity and the second bearing capacity of the soil.

18. The vehicle of claim 17 wherein the vehicle system comprises at least one of:
a tire pressure system inflating or deflating tires of the vehicle based on the bearing capacity,
a differential engaging or disengaging based on the bearing capacity;
a traction control system controlling the vehicle based on the bearing capacity; and
a drive mode selector selecting a drive mode based on the bearing capacity.

19. The vehicle of claim 17 wherein the first rotating penetrometer and the second rotating penetrometer each comprise:
a circuit board;
a plurality of strain sensors disposed on the circuit board generating strain signals;
an accelerometer disposed on the circuit board generating an acceleration signal;
a gyroscopic sensor generating an orientation signal;
a load cell coupled to an actuator arm; and
a moisture sensor.

20. A method of controlling a vehicle in soil comprising,
rotating a penetrometer on the soil;
generating sensor signals from the penetrometer during rotating;
determining a sheer strength and bearing capacity based on the sensor signals; and
controlling a vehicle system based on the bearing capacity and shear strength.
